(19) United States
(12) Patent Application Publication
(10) Pub. No.: US 2009/0069562 A1
(43) Pub. Date: Mar. 12, 2009

(54) Process for the preparation of alfuzosin

(76) Inventors: Joseph Prabahar Koilpillai, Andhra Pradesh (IN); Magesh Subramanian, Andra Pradesh (IN); Uppalalah Mallela, Andra Pradesh (IN); Sivakumaran Meenakshlsunderam, Andra Pradesh (IN)

Correspondence Address: Jay R Akhave, 2058 N Mills Ave, # 612, Claremont, CA, 91711, US

(21) Appl. No.: 11/667,209

(22) PCT Filed: Dec. 11, 2006
(86) PCT No.: PCT/IB06/03643

§ 371 Date: May 7, 2007

(30) Foreign Application Priority Data
Dec. 26, 2005 (IN) 1912/CHE/2005

(51) Int. Cl. C07D 239/72

(52) U.S. Cl. 544/292

(57) ABSTRACT

An improved process for the preparation of N-[3-[(4-amino-6,7-dimethoxy-2-quinazolinyl)methylamino]propyl]tetrahydrofuran-2-carboxamide of Formula (I),

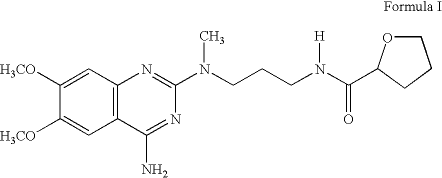

by reacting N₁-(4-amino-6,7-dimethoxyquinazolin-2-yl)-N₁-methyl propane-1,3-diamine (V)

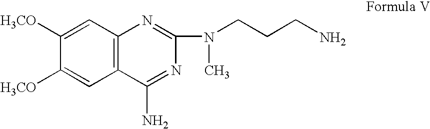

with tetrahydrofuran-2-carboxylic acid or its reactive derivative in a solvent.

### FIELD OF THE INVENTION

The present invention relates to an improved process for the preparation of N-[3-[(4-amino-6,7-dimethoxy-2-quinazolinyl)methylamino]propyl]tetrahydrofuran-2-carboxamide of Formula (I).

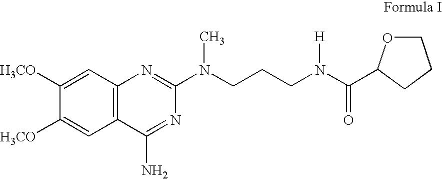

### BACKGROUND OF THE INVENTION

N-[3-[(4-Amino-6,7-dimethoxy-2-quinazolinyl)methylamino]propyl]tetrahydrofuran-2-carboxamide, is generically known as Alfuzosin. Alfuzosin is an α₁-Adrenoceptor antagonist and the hydrochloride salt of Alfuzosin is approved for the treatment of Benign Prostatic Hyperplasia and is marketed in the US with the Brand Name, UROXATRAL.

Synthelabo, first time disclosed Alfuzosin and its pharmaceutically acceptable salts in U.S. Pat. No. 4,315,007. In this patent, Alfuzosin hydrochloride is found to have anti-hypertensive activity. Subsequently, the use of Alfuzosin hydrochloride as an anti-dysuritic agent, for the treatment of Benign Prostatic Hyperplasia is claimed in U.S. Pat. No. 4,661,491.

U.S. Pat. No. 4,315,007 describes two different synthetic routes for the preparation of Alfuzosin. One of the synthetic routes involves reaction of 4-amino-2-chloro-6,7-dimethoxyquinazoline (II) with 3-methylaminopropionitrile (III) in isoamyl alcohol to produce 3-[(4-amino-6,7-dimethoxy-2-quinazolinyl)methylamino]propanenitrile (IV). The propanenitrile compound (IV) is hydrogenated using Raney Nickel in an alcoholic solvent in presence of ammonia to produce N₁-(4-amino-6,7-dimethoxyquinazolin-2-yl)-N₁-methylpropane-1,3-diamine (V), which is converted to Alfuzosin by reaction with tetrahydrofuran-2-carboxylic acid. Alfuzosin is further converted to its hydrochloride salt by treatment with ethanolic hydrogen chloride. This process is shown in the Scheme given below:

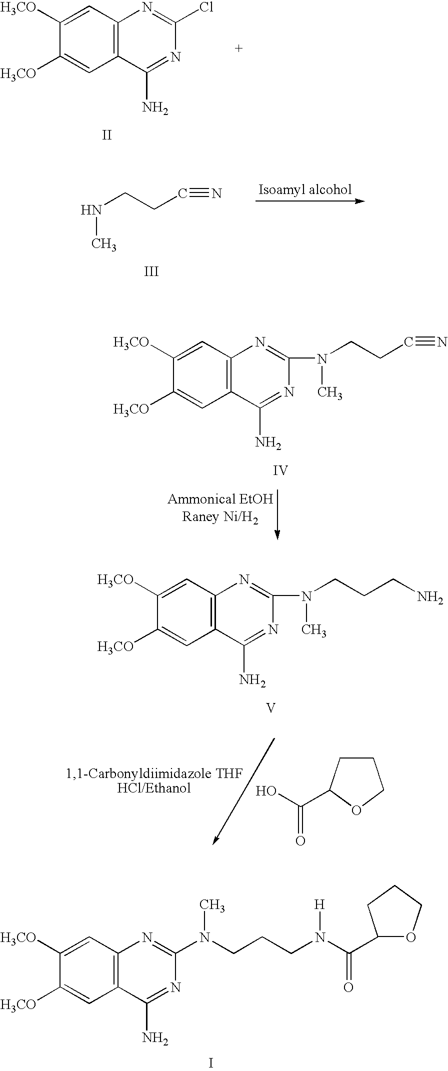

The above process is also disclosed in J. Med. Chem., 1996, 29(1), 19-25, with a slight variation, as shown below:

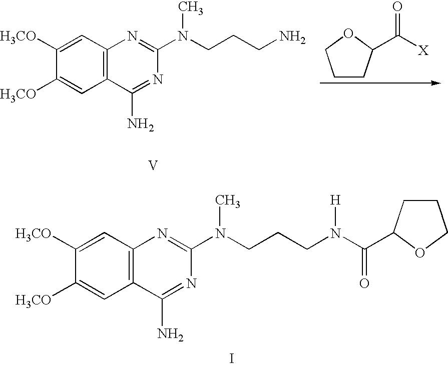

wherein the group X is not specifically defined.

The major disadvantage with the above prior-art process is the low yield of 3-[(4-amino-6,7-dimethoxy-2-quinazolinyl)methylamino]propanenitrile (IV), which is the key intermediate in the preparation of Alfuzosin. The yield of compound (v) reported in J. Med. Chem., 1996, 29(1), 19-25 is 62%. No reason has been attributed to this low yield of compound (IV) in the prior-art. We have now found that the cause of this low yield is due to the formation of an undesired compound (IVA). During preparation of compound (IV) as per the prior art procedure, the compound (IVA) is formed to the extent of 20%, because of competing Retro-Michael reaction. It requires repeated purification to remove the impurity (IVA), which results in low yield of compound (IV).

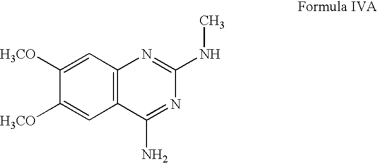

Further, another disadvantage of the above prior art process of preparing Alfuzosin is the use of expensive and highly moisture sensitive reagent, 1,1-carbonyldiimidazole, during acylation. Use of this reagent on industrial scale is not preferred due to anhydrous conditions required in the process.

In the instant invention, it has been found that the reaction of 4-amino-2-chloro-6,7-dimethoxyquinazoline (II) with 3-methylaminopropionitrile (III) in presence of an acidic reagent, controls the formation of Retro-Michael impurity of Formula IVA, thereby resulting in higher yield of compound (IV) in the range of 90% and above. Further, the conversion of diamine compound (V) to Alfuzosin has been achieved without the application of an expensive and moisture sensitive 1,1-carbonyldiimidazole.

### OBJECTIVE OF THE INVENTION

The main objective of the present invention is to provide a simple and effective process for the preparation of Alfuzosin with high purity and good yields on a commercial scale.

### SUMMARY OF THE INVENTION

Accordingly, the present invention relates to an improved process for the preparation of N-[3-[(4-amino-6,7-dimethoxy-2-quinazolinyl)methylamino]propyl]tetrahydrofuran-2-carboxamide (Alfuzosin) of Formula (I),

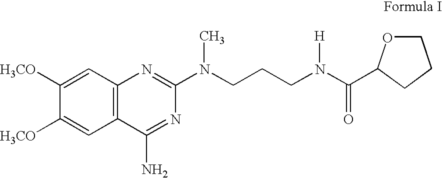

and its pharmaceutically acceptable salts, by reacting N₁-(4-amino-6,7-dimethoxyquinazolin-2-yl)-N₁-methylpropane-1,3-diamine (V)

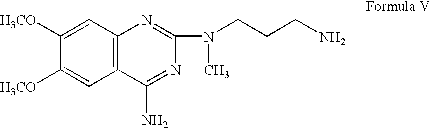

with tetrahydrofuran-2-carboxylic acid or its reactive derivative.

According to another embodiment of the present invention, the N₁-(4-amino-6,7-dimethoxyquinazolin-2-yl)-N₁-methylpropane-1,3-diamine (V) is prepared by condensing 4-amino-2-chloro-6,7-dimethoxyquinazoline (II)

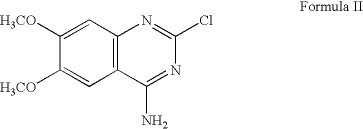

with 3-methylaminopropionitrile (III), in presence of an acid reagent, in an alcoholic solvent to produce 3-[(4-amino-6,7-dimethoxy-2-quinazolinyl)methylamino]propanenitrile (IV),

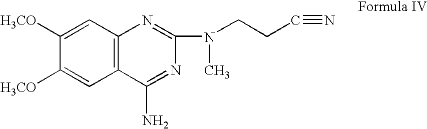

and hydrogenating the intermediate (IV) in presence of metal catalyst in a solvent to produce N₁-(4-amino-6,7-dimethoxyquinazolin-2-yl)-N₁-methylpropane-1,3-diamine (V).

### DETAILED DESCRIPTION OF THE INVENTION

N₁-(4-Amino-6,7-dimethoxyquinazolin-2-yl)-N₁-methylpropane-1,3-diamine (V) is prepared in high yield and good purity from 4-amino-2-chloro-6,7-dimethoxyquinazoline (II) and 3-methylaminopropionitrile (III) in presence of an acidic reagent. The acidic reagent is selected from sulfonic acids such as p-toluenesulfonic acid, methanesulfonic acid, benzenesulfonic acid or trifluoromethanesulfonic acid and most preferably p-toluenesulfonic acid, phenol and substituted phenols, such as p-nitrophenol. The reaction is carried out in an alcoholic solvent selected from isoamyl alcohol, 1-butanol, 1-propanol, 2-propanol, ethanol, 2-methoxyethanol and most preferably ethanol at a temperature of about 50 to 80° C. The compound (IV) is hydrogenated using metal catalyst selected from Raney Nickel, Palladium/Carbon, Rhodium alumina and most preferably Raney Nickel in a solvent selected from ammonical methanol, ammonical ethanol at 1 to about 20 kg of hydrogen pressure at a temperature of about 30° C. to 80° C.

The acylation of N₁-(4-amino-6,7-dimethoxyquinazolin-2-yl)-N₁-methylpropane-1,3-diamine (V) with tetrahydrofuran-2-carboxylic acid or its reactive derivative is carried out in aprotic organic solvents like halogenated hydrocarbons, toluene, alkyl esters, alkyl ethers etc, but the preferred solvent is methylenechloride, at a temperature of about −5° C. to 10° C.

When the reaction is carried out with tetrahydrofuran-2-carboxylic acid, a condensing agent such as N,N′-dicyclohexylcarbodiimide alone or in combination with 1-hydroxybenzotriazole is used.

When the reaction is carried out with the reactive derivative of tetrahydrofuran-2-carboxylic acid, the reactive derivative is selected from an acid anhydride, mixed acid anhydrides, reactive esters, and reactive amides. The activation is carried with ethyl chloroformate, methyl chloroformate or pivaloyl chloride in aprotic organic solvent in presence of an organic base selected from triethylamine, diethylamine, tributylamine, N-alkylanilines, 1,8-diazabicyclo[5.4.2]undec-7-ene, 1,5-diazabicyclo[4.3.0]non-5-ene, N-methylmorpholine, 1,4-diazabicyclo[2.2.2]octane, 4-dimethylaminopyridine and mixtures thereof.

After the completion of the reaction, as ascertained by the known detection methods reported in the art, water is added to the reaction mixture and pH is adjusted to 4.0 to 5.0 with an acid. The organic layer is separated and the pH of the aqueous layer is further adjusted to about 10.0 to 10.5 with an inorganic base selected from aqueous sodium hydroxide, aqueous potassium hydroxide or aqueous ammonia. Alfuzosin is extracted into a water immiscible organic solvent and is isolated from acetone, acetonitrile, methanol, ethanol, 2-propanol, diisopropylether etc.

Alfuzosin prepared according to the process of the present invention is converted to its pharmaceutically acceptable salts such as hydrochloride, by methods reported in the prior-art.

The following examples to prepare Alfuzosin illustrate the nature of the invention and are provided for illustrative purpose only and should not be construed to limit the scope of the invention.

### EXAMPLE 1

### Stage I

### Preparation of N₁-(4-amino-6,7-dimethoxyquinazolin-2-yl)-N₁-methylpropane-1,3-diamine (V)

### A) Preparation of 3-[(4-amino-6,7-dimethoxy-2-quinazolinyl)methylamino]-propanenitrile (IV)

A mixture of 4-amino-2-chloro-6,7-dimethoxyquinazoline (50 g, 0.208 mol), 3-methylaminopropionitrile (35.07 g, 0.416 mol) and p-toluenesulphonic acid (39.71 g, 0.208 mol) was heated to reflux in 1-butanol (350 ml) for 3 hr. The reaction mass was concentrated at 70-80° C. under reduced pressure. The resulting concentrated mass was stirred with aqueous ethanol (50% v/v, 250 ml) and pH of the mixture was adjusted to 10.0 to 10.5. Thereafter, the precipitate was filtered and dried at 60-65° C. under vacuum to obtain 3-[(4-amino-6,7-dimethoxy-2-quinazolinyl)methylamino]-propanenitrile. Yield: 55.0 g (92% of theory)

### B) Preparation of N₁-(4-amino-6,7-dimethoxyquinazolin-2-yl)-N₁-methylpropane-1,3-diamine (V)

3-[(4-Amino-6,7-dimethoxy-2-quinazolinyl)methylamino]propanenitrile (50 g) was dissolved in 15% ammonical ethanol and hydrogenated in presence of Raney nickel at 50-55° C. under 10-15 kg hydrogen pressure. After completion of reduction, catalyst was removed by filtration and the filtrate was concentrated at 40-45° C. under reduced pressure. This concentrated mass was crystallized from methanol.

### Stage II

### Preparation of N-[3-[(4-amino-6,7-dimethoxy-2-quinazolinyl)methylamino]propyl]tetrahydrofuran-2-carboxamide (Alfuzosin)

Ethyl chloroformate (22.35 g, 0.206 mol) was added to a mixture of tetrahydrofuran-2-carboxylic acid (23.90 g, 0.206 mol), and triethylamine (20.80 g, 0.206 mol) in methylene dichloride (300 ml) at 0-5° C. The stirring was continued for 30 min at 0-5° C. to complete the formation of mixed anhydride. To this, mixture of N₁-(4-amino-6,7-dimethoxyquinazolin-2-yl)-N₁-methylpropane-1,3-diamine (50 g, 0.172 mol) in methylene dichloride (200 ml) was added at 0-5° C. and stirring was continued for additional 1 hr at 0-5° C. to complete the reaction. Thereafter, water was added to the reaction mass and pH was adjusted to 4.0-4.5. The organic layer was discarded and the pH of the aqueous layer was raised to 10-10.5 with aqueous sodium hydroxide. The aqueous layer was extracted with methylene dichloride and the organic extract was concentrated to remove methylene dichloride. The concentrated mass was stirred with acetone to afford Alfuzosin. The product was filtered and dried under vacuum. Yield: 50 g (75% of theory). HPLC purity: 99.97%.

### EXAMPLE 2

### Preparation of N-[3-[(4-amino-6,7-dimethoxy-2-quinazolinyl)methylamino]propyl]tetrahydrofuran-2-carboxamide (Alfuzosin)

A mixture of N,N′-dicyclohexylcarbodiimide (39.96 g, 0.206 mol) and tetrahydrofuran-2-carboxylic acid (23.90 g, 0.206 mole) was stirred in methylene dichloride (500 ml) at 0-5° C. To this mixture, N₁-(4-amino-6,7-dimethoxyquinazolin-2-yl)-N₁-methylpropane-1,3-diamine (50 g, 0.172 mol) was added and continued stirring at 0-5° C. for 1 hr. The by-product, dicyclohexylurea was filtered. To the filtrate, water was added and adjusted the pH of the mixture to 4.0-4.5. The organic layer was discarded and the pH of the aqueous layer was raised to 10-10.5 with aqueous sodium hydroxide. Aqueous layer was extracted with methylene dichloride and the methylene dichloride extract was concentrated. The oily mass was stirred with acetone to afford Alfuzosin. The product was filtered and dried under vacuum. Yield: 49 g (73% of theory). HPLC purity: 99.39%.

### EXAMPLE 3

### Preparation of N₁-[3-[(4-amino-6,7-dimethoxy-2-quinazolinyl)methyl-aminopropyl]tetrahydrofuran-2-carboxamide (Alfuzosin)

Pivaloyl chloride (20.7 g, 0.172 mol) was added to a mixture of tetrahydro-2-furoic acid (21.92 g, 0.189 mol) and triethylamine (19.10 g, 0.189 mol) in methylene chloride (300 ml) at −10 to −5° C. Stirring was continued for 30 min at −10 to −5° C. to complete the formation of mixed anhydride. To this mixture, N₁-(4-amino-6,7-dimethoxyquinazolin-2-yl)-N₁-methylpropane-1,3-diamine (50 g, 0.172 mol) was added and stirring was continued for an additional 1 h at −10 to −5° C. to complete the reaction. Thereafter, water was added to the reaction mass and pH was adjusted to 4.0-4.5. The organic layer was discarded and the pH of the aqueous layer was raised to 10-10.5 with aqueous sodium hydroxide solution. The aqueous layer was extracted with methylene chloride and the organic extract was concentrated to remove methylene chloride. The concentrated mass was stirred with acetonitrile at 50-55° C. to afford Alfuzosin base. The product was filtered and dried under vacuum.

YIELD: 52.5 g (78.5% of theory), HPLC PURITY:99.97%.

We claim

1. An improved process for the preparation of N-[3-[(4-amino-6,7-dimethoxy-2-quinazolinyl)methylamino]propyl]tetrahydrofuran-2-carboxamide of Formula (I) 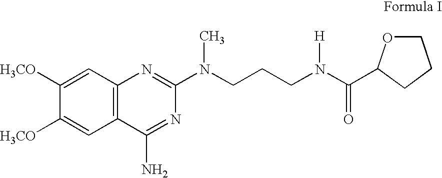
which comprises reacting N₁-(4-amino-6,7-dimethoxyquinazolin-2-yl)-N₁-methyl propane-1,3-diamine (V) 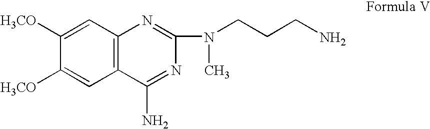
with tetrahydrofuran-2-carboxylic acid or its reactive derivative in presence of condensing agent selected from N,N′-dicyclohexylcarbodiimide in a solvent.

2. The process according to claim 1, wherein a condensing agent is used in combination with 1-hydroxybenzotriazole when the reaction is carried out with tetrahydrofuran-2-carboxylic acid.

3. The process according to claim 1, wherein the reactive derivative is selected from an acid anhydride, mixed acid anhydrides, reactive esters, and reactive amides.

4. The process according to claim 3, wherein the reactive derivative is prepared by activation with ethyl chloroformate, methyl chloroformate or pivaloyl chloride, in presence of an organic base.

5. The process according to claim 4, wherein the organic base is selected from triethylamine, diethylamine, tributylamine, N-alkylanilines, 1,8-diazabicyclo[5.4.2]undec-7-ene, 1,5-diazabicyclo[4.3.0]non-5-ene, N-methylmorpholine, 1,4-diazabicyclo[2.2.2]octane, 4-dimethylaminopyridine and mixtures thereof.

6. The process according to claim 1, wherein the reaction is carried out in aprotic organic solvents such as halogenated hydrocarbons, toluene, alkyl esters or alkyl ethers.

7. A process according to claim 1, wherein N₁-(4-amino-6,7-dimethoxyquinazolin-2-yl)-N₁-methylpropane-1,3-diamine (V) is prepared by condensing 4-amino-2-chloro-6,7-dimethoxyquinazoline (II) 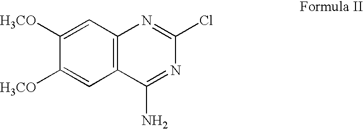
with 3-methylaminopropionitrile (III) in presence of an acidic reagent in a solvent to produce 3-[(4-amino-6,7-dimethoxy-2-quinazolinyl)methylamino]propane-nitrile (IV), 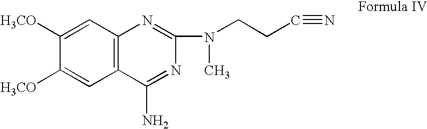
and hydrogenating the compound of Formula IV in presence of a metal catalyst.

8. The process according to claim 7, wherein the acidic reagent is selected from sulfonic acids such as p-toluenesulfonic acid, methanesulfonic acid, benzenesulfonic acid or trifluoromethanesulfonic acid.

9. The process according to claim 7, wherein the acidic reagent is selected from phenol and substituted phenols, such asp-nitrophenol.

10. The process according to claim 7, wherein the solvent is selected from isoamyl alcohol, 1-butanol, 1-propanol, 2-propanol, ethanol, 2-methoxyethanol.
